(19) United States
(12) Patent Application Publication
(10) Pub. No.: US 2015/0094612 A1
(43) Pub. Date: Apr. 2, 2015

(54) MEDICAL INSTRUMENT

(71) Applicant: TERUMO KABUSHIKI KAISHA, Shibuya-ku (JP)

(72) Inventors: Kaoru MIYAZAKI, Fujinomiya-shi (JP); Wataru KARINO, Atsugi-shi (JP)

(73) Assignee: TERUMO KABUSHIKI KAISHA, Tokyo (JP)

(21) Appl. No.: 14/566,076

(22) Filed: Dec. 10, 2014

Related U.S. Application Data
(63) Continuation of application No. PCT/JP2013/065345, filed on Jun. 3, 2013.

(30) Foreign Application Priority Data
Jun. 11, 2012 (JP) 2012-132196

(51) Int. Cl. A61B 10/06; A61B 10/02

(52) U.S. Cl. CPC A61B 10/06; A61B 10/0266
USPC 600/564; 600/562

(57) ABSTRACT

A medical instrument is disclosed, which includes an elongated member having a plurality of joint members arranged side by side in an axial direction, the elongated member taking a first form that is bendable in a direction intersecting with the axial direction when the joint members are arranged with gaps therebetween in the axial direction and taking a second form having rigidity when the distance between the joint members is reduced in the axial direction to be narrower than the distance in the first form, a gripper capable of gripping an object to be gripped, and a first push-pull member and a second push-pull member each movable along the axial direction of the elongated member. Operating the first push-pull member and the second push-pull member enables the elongated member to be switched between the bendable first form and the rigid second form and enables an opening-closing operation of the gripper.

### CROSS-REFERENCES TO RELATED APPLICATIONS

This application is a continuation of International Application No. PCT/JP2013/065345 filed on Jun. 3, 2013, and claims priority to Japanese Application No. 2012-132196 filed on Jun. 11, 2012, the entire content of both of which is incorporated herein by reference.

### TECHNICAL FIELD

The present disclosure generally relates to a medical instrument used for collecting living tissue.

### BACKGROUND DISCUSSION

There is known, as a medical instrument which collects living tissue, a biopsy forceps provided with a gripper which grips living tissue inside a living body and an elongated member for introducing the gripper into the living body. In known biopsy forceps, an operation member for opening and closing the gripper is disposed on the hand side of the elongated member. Further, the elongated member can be flexible to make the elongated member bendable in order to improve the deliverability of the gripper to various sites of a living organ.

For example, living tissue present within the wall of a living organ may be collected using such a biopsy forceps. However, when the elongated member has flexibility as described above, the pushability from the hand side to the gripper can be low. Thus, it may not be possible to insert the gripper into the wall, and to collect the living tissue.

JP 2007-511248 W describes a medical elongated member that is switchable between a bendable state and a rigid state. The elongated member is provided with a plurality of joint members and a push-pull member, which couples the joint members to each other. The push-pull member is operated to be pulled or pushed back to increase or reduce the distance between the joint members, which enables the elongated member to be switched between the bendable state and the rigid state. When such an elongated member is applied to an elongated member of a biopsy forceps, the elongated member can have bendability when the elongated member moves inside a living body and can have rigidity when the gripper is inserted into the wall of a living organ. Therefore, biopsy forceps capable of collecting living tissue within the wall without deteriorating the deliverability of the gripper can be provided.

### SUMMARY

When using a biopsy forceps into which the elongated member provided with the push-pull member as described above is incorporated, it can be necessary to switch the elongated member between the bendable state and the rigid state through the push-pull member and also necessary to operate an opening-closing operation of the gripper through an operation member which is provided separately from the push-pull member, which can complicate the configuration of the device. In addition, this can complicate the operation of the medical instrument and also result in poor operability of the medical instrument.

In view of the above, a medical instrument is disclosed having excellent operability, and which is capable of collecting living tissue present within the wall of a living organ without deteriorating the deliverability of a gripper inside a living body.

In accordance with an exemplary embodiment, a medical instrument is disclosed, which can include an elongated member having a plurality of joint members arranged side by side in an axial direction, the elongated member taking a first form that is bendable in a direction intersecting with the axial direction when the joint members are arranged with gaps therebetween in the axial direction and taking a second form having rigidity when a distance between the joint members is reduced in the axial direction to be narrower than the distance in the first form; a gripper arranged on one end side of the elongated member, the gripper having a first open-close member and a second open-close member, the first open-close member and the second open-close member being movable closer to each other or away from each other; a first push-pull member coupled to the joint members and the first open-close member, the first push-pull member being movable along the axial direction of the elongated member; and a second push-pull member coupled to the joint members at positions differing from positions at which the first open-close member is coupled and to the second open-close member, the second push-pull member being movable along the axial direction of the elongated member, wherein the elongated member takes the second form by pulling the first push-pull member and the second push-pull member in the axial direction with the elongated member taking the first form to reduce the distance between the joint members, a first operation for opening or closing the gripper is driven by pulling the first push-pull member and the second push-pull member with the elongated member taking the second form, the elongated member takes the first form by pushing back the first push-pull member and the second push-pull member to move in a direction opposite to the pulling direction after the first operation of the gripper has been driven to increase the distance between the joint members, and a second operation that is opposite to the first operation for opening or closing the gripper is driven by pushing back the first push-pull member and the second push-pull member to move in the direction opposite to the pulling direction with the elongated member taking the first form.

In accordance with an exemplary embodiment, a medical instrument is disclosed, which can further include a first auxiliary push-pull member coupled to the first open-close member, the first auxiliary push-pull member being movable along the axial direction of the elongated member; and a second auxiliary push-pull member coupled to the second open-close member, the second auxiliary push-pull member being movable along the axial direction of the elongated member, wherein an opening-closing operation of the gripper can be driven by pulling the first auxiliary push-pull member and the second auxiliary push-pull member along the axial direction or pushing back the first auxiliary push-pull member and the second auxiliary push-pull member to move in a direction opposite to the pulling direction.

In accordance with an exemplary embodiment, a medical instrument is disclosed, wherein each of the first open-close member and the second open-close member has a tapered shape tapered toward a tip side, and blades are formed on gripping surfaces on which the first and second open-close members face each other.

In accordance with an exemplary embodiment, a medical instrument is disclosed, wherein the joint members have through holes penetrating the elongated member in the axial direction.

In accordance with an exemplary embodiment, a medical instrument is disclosed, further including a displacement preventer that is deformed to project in a direction intersecting with the axial direction of the elongated member when the elongated member is switched from the first form to the second form to prevent the elongated member from being displaced from a predetermined position.

In accordance with an exemplary embodiment, a medical instrument is disclosed, further including a hand operation unit arranged on the other end side of the elongated member, the hand operation unit being capable of operating movement of the first push-pull member and the second push-pull member, wherein the hand operation unit has an open-close holder restricting the movement of the first push-pull member and the second push-pull member to hold the gripper in an open state or a closed state.

In accordance with an exemplary embodiment, a medical instrument is disclosed, wherein the first operation moves the first open-close member and the second open-close member away from each other to open the gripper, and the second operation moves the first open-close member and the second open-close member closer to each other to close the gripper.

In accordance with an exemplary embodiment, a medical instrument is disclosed, wherein the medical instrument is a living tissue collecting medical instrument used for collecting living tissue in a respiratory region.

In accordance with an exemplary embodiment, a medical instrument is disclosed, wherein the elongated member can be switched between the bendable first form and the second form having rigidity and also to open and close the gripper by performing the operation of pulling the first push-pull member and the second push-pull member and the operation of pushing back the first push-pull member and the second push-pull member to move in the direction opposite to the pulling direction. Thus, it is not necessary to provide an operation member for operating the opening-closing operation of the gripper in addition to the push-pull members for operating the switching of the elongated member between the bendable state and the rigid state. Accordingly, the configuration of the device can be prevented from becoming complicated. Therefore, the medical instrument can have excellent operability, and being capable of collecting living tissue present within the wall of a living organ without deteriorating the deliverability of the gripper inside a living body.

In accordance with an exemplary embodiment, a medical instrument is disclosed, wherein the gripper can be directly opened and closed by operating the first auxiliary push-pull member and the second auxiliary push-pull member. Therefore, the gripper can be reliably opened and closed.

In accordance with an exemplary embodiment, a medical instrument is disclosed, wherein the gripper can be smoothly inserted into the wall of a living organ. Further, an object to be gripped within the wall by cutting the object can be collected.

In accordance with an exemplary embodiment, a medical instrument is disclosed, wherein the lumen which penetrates the joint members can be arranged side by side on the elongated member.

In accordance with an exemplary embodiment, a medical instrument is disclosed, wherein the elongated member can be prevented from being displaced when the elongated member is switched from the first form to the second form.

In accordance with an exemplary embodiment, a medical instrument is disclosed, wherein various operations can be performed while holding the gripper in an open state or a closed state by the open-close holder provided in the hand operation unit.

In accordance with an exemplary embodiment, a medical instrument is disclosed, wherein the operation of pulling the push-pull members is converted into the operation of opening the gripper. Thus, the gripper can be opened with a stronger force than when the gripper is opened by pushing back the push-pull members. Therefore, the gripper can be reliably opened in the wall of a living organ.

In accordance with an exemplary embodiment, a medical instrument is disclosed, wherein the living tissue collecting medical instrument can be suitable for collecting living tissue in a respiratory region.

### BRIEF DESCRIPTION OF THE DRAWINGS

FIG. 1 is a diagram illustrating, in a simplified manner, the entire configuration of a medical instrument according to an exemplary embodiment of the present disclosure.

FIGS. 2A to 2C are diagrams illustrating the configuration of a gripper of the medical instrument according to the exemplary embodiment, wherein FIG. 2A is an enlarged view illustrating the gripper in a closed state, FIG. 2B is an enlarged view illustrating the gripper in an open state, and FIG. 2C is a perspective view of the appearance of the gripper in an open state.

FIGS. 3A and 3B are diagrams illustrating the configuration of a joint member of the medical instrument according to the embodiment, wherein FIG. 3A is a front view of the joint member and FIG. 3B is a top view of the joint member.

FIGS. 4A and 4B are top views of joint members according to modifications.

FIGS. 5A and 5B are diagrams illustrating a pulling mechanism of the medical instrument according to the embodiment, wherein FIG. 5A illustrates a pulling mechanism which pulls push-pull members by a rotation operation, and FIG. 5B illustrates a pulling mechanism which pulls the push-pull members by a slide operation.

FIG. 6 is a diagram illustrating the action of the medical instrument according to an exemplary embodiment, for example, a diagram illustrating a state in which the medical instrument with an elongated member in a bendable first form has been introduced into a living body.

FIG. 7 is a diagram illustrating the action of the medical instrument according to an exemplary embodiment, for example, a diagram illustrating a state in which the gripper of the medical instrument has been inserted into the wall of a living organ.

FIG. 8 is a diagram illustrating the action of the medical instrument according to an exemplary embodiment, for example, a diagram illustrating a state in which the gripper has been opened from the state illustrated in FIG. 7.

FIG. 9 is a diagram illustrating the action of the medical instrument according to an exemplary embodiment, for example, a diagram illustrating a state in which the gripper has been pushed into the wall of the living organ from the state illustrated in FIG. 8.

FIG. 10 is a diagram illustrating the action of the medical instrument according to the embodiment, for example, a diagram illustrating a state in which the push-pull members have been pushed back from the state illustrated in FIG. 9 to bring the elongated member into the bendable first form.

FIG. 11 is a diagram illustrating the action of the medical instrument according to the embodiment, for example, a diagram illustrating a state in which the gripper has been closed from the state illustrated in FIG. 10.

### DETAILED DESCRIPTION

Set forth below, an exemplary embodiment of the present disclosure will be described with reference to the drawings. For convenience of explanation, the dimensional ratio in each of the drawings is exaggerated and may therefore differ from an actual ratio.

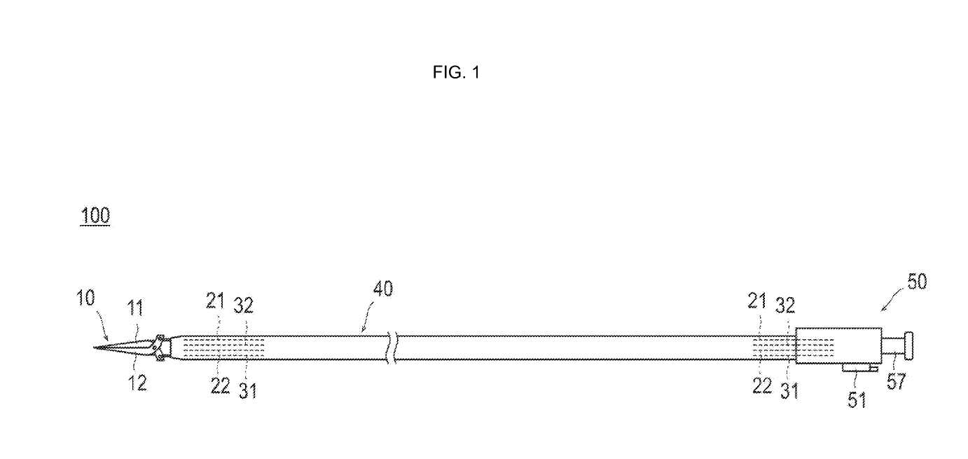

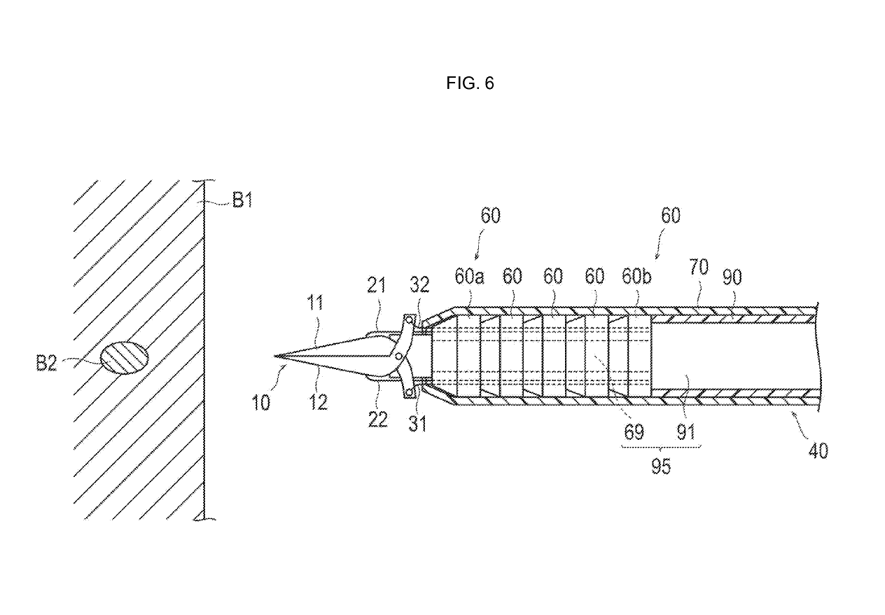

FIGS. 1 to 5B are diagrams illustrating configurations of a medical instrument according to an exemplary embodiment. FIGS. 6 to 11 are diagrams illustrating the action of the medical instrument according to an exemplary embodiment.

As illustrated in FIG. 1, a medical instrument 100 is disclosed, which can include an elongated member 40 which can be introduced into a living body, a gripper 10 which is arranged on one end of the elongated member 40, and a hand operation unit 50 which is arranged on the other end of the elongated member 40. In the description of this specification, one end (the left side in FIG. 1) on which the gripper 10 is arranged is also referred to as a tip, and the other end (the right side in FIG. 1) on which the hand operation unit 50 is arranged is also referred to as a base end.

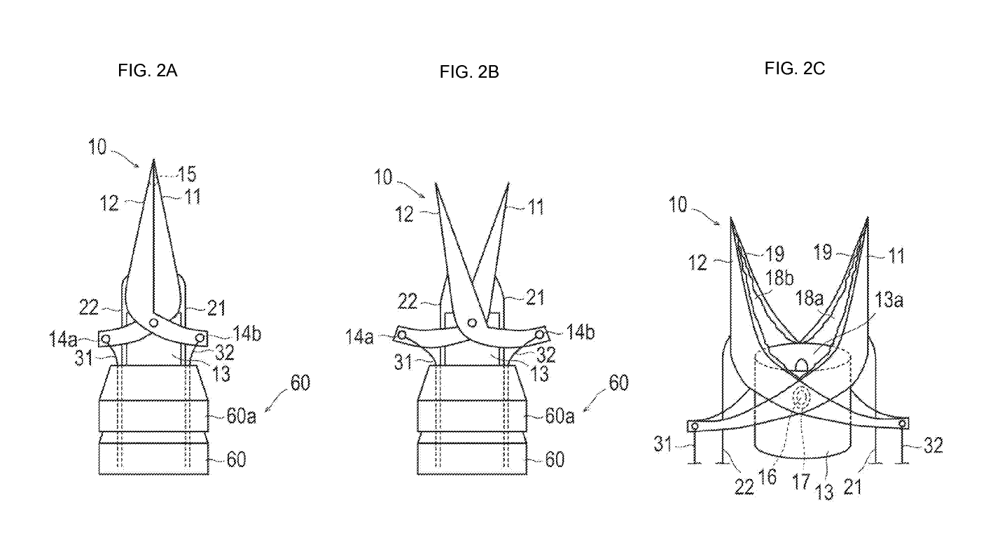

As illustrated in FIGS. 2A to 2C, the gripper 10 can include a first open-close member 11 and a second open-close member 12. The first open-close member 11 and the second open-close member 12 have a configuration capable of performing an opening-closing operation for gripping an object to be gripped by moving closer to each other and releasing the gripped object by moving away from each other. The opening-closing operation can be driven by operating a first push-pull member 21, which is coupled to the first open-close member 11 and a second push-pull member 22, which is coupled to the second open-close member 12.

The first push-pull member 21 is provided movably back and forth along the axial direction of the elongated member 40. When the first push-pull member 21 is pulled towards the base end side, the first push-pull member 21 pulls the first open-close member 11 in response to the pulling operation to thereby move the first open-close member 11 away from the second open-close member 12. When the first push-pull member 21 is pushed towards the tip side, the first open-close member 11 moves closer to the second open-close member 12 in a direction opposite to that when the first push-pull member 21 is pulled. The second push-pull member 22 also operates in the same manner as the first push-pull member 21. For example, when the second push-pull member 22 is pulled towards the base end side, the second push-pull member 22 moves the second open-close member 12 away from the first open-close member 11. When the second push-pull member 22 is pushed towards the tip side, the second push-pull member 22 moves the second open-close member 12 closer to the first open-close member 11. Thus, when the first push-pull member 21 and the second push-pull member 22 are pulled toward the base end side, the first open-close member 11 and the second open-close member 12 move away from each other to open the gripper 10. When the first push-pull member 21 and the second push-pull member 22 are pushed toward the tip side, the first open-close member 11 and the second open-close member 12 move closer to each other to close the gripper 10.

The first push-pull member 21 and the second push-pull member 22 may be composed of, for example, flexible string-like elongated members, or may also be composed of flexible plate-like members. In the medical instrument 100, the first push-pull member 21 and the second push-pull member 22 can be made of nickel titanium. However, the material of the first push-pull member 21 and the second push-pull member 22 is not particularly limited. For example, a material that enables a predetermined force to be transmitted to the first open-close member 11 and the second open-close member 12 through the first push-pull member 21 and the second push-pull member 22 may be appropriately selected.

In accordance with an exemplary embodiment, for example, the first push-pull member 21 and the second push-pull member 22 may be directly coupled to the outer faces of the lateral sides of the first open-close member 11 and the second open-close member 12, respectively. A method of the coupling is not particularly limited. For example, methods such as adhesion, welding, and soldering may be employed.

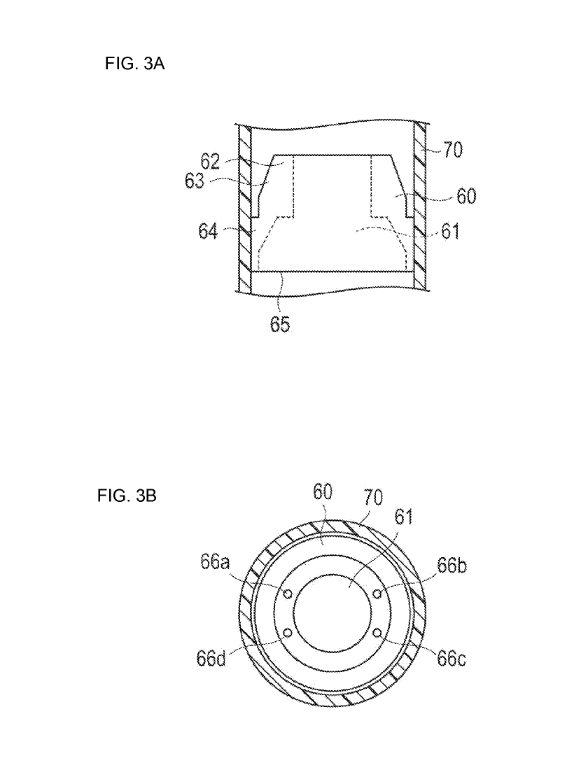

The first open-close member 11 and the second open-close member 12 can be rotatably attached to a hollow base 13 which has a through hole 13a penetrating the base 13 in the axial direction and is arranged on the tip side of the elongated member 40 (refer to FIG. 2C). The base 13 constitutes a tip part of the elongated member 40 (refer to FIG. 6). The through hole 13a of the base 13 constitutes a tip part of a lumen 95 which is formed on the elongated member 40. The base 13 may be a member independent from a joint member 60 of the elongated member 40 (described below, refer to FIGS. 3A, 3B, and 6), or may also be a member integrated with the joint member 60.

The first open-close member 11 and the second open-close member 12 are attached to the base 13, for example, in such a manner that projections 16, each of which functions as a rotation shaft are formed on the first open-close member 11 and the second open-close member 12, recesses 17 to which the projections 16 can be fitted are formed on the base 13, and the projections 16 are fitted into the recesses 17.

The first open-close member 11 and the second open-close member 12 may have, for example, a tapered shape whose outer shape is tapered toward the tip side. A blade 19 may be formed on a gripping surface 18a of the first open-close member 11, the gripping surface 18a facing the second open-close member 12. In addition, a blade 19 may be formed on a gripping surface 18b of the second open-close member 12, the gripping surface 18b facing the first open-close member 11.

In the medical instrument 100, separately from the first push-pull member 21 and the second push-pull member 22, a first auxiliary push-pull member 31 and a second auxiliary push-pull member 32 each capable of independently driving the opening-closing operation of the gripper 10 may be provided in addition to the first push-pull member 21 and the second push-pull member 22. The first auxiliary push-pull member 31 may be coupled to the first open-close member 11, for example, through a coupling hole 14a formed on an end of the first open-close member 11. The second auxiliary push-pull member 32 may be coupled to the second open-close member 12, for example, through a coupling hole 14b formed on an end of the second open-close member 12. As the material, configuration, and coupling method to the respective open-close members of the first auxiliary push-pull member 31 and the second auxiliary push-pull member 32, the same material, configuration, and coupling method as in the first push-pull member 21 and the second push-pull member 22 may be employed.

As illustrated in FIGS. 2A to 3B, the elongated member 40 of the medical instrument 100 can include a plurality of joint members 60 which are arranged side by side. The plurality of joint members 60 arranged along the axial direction makes the elongated member 40 selectively deformable between a bent state and a rigid state. As illustrated in FIG. 6, the elongated member 40 may be provided with an inner insertion tube 90 which is arranged to have contact with a joint member 60b located on the base end side and an outer fitting tube 70 which is arranged to cover the joint members 60 and the inner insertion tube 90. As will be described below, the inner insertion tube 90 is a member used for restricting movement of the joint members 60 toward the base end side when the distance between the joint members 60 is reduced. The outer fitting tube 70 is a member used for protecting the joint members 60 and the like of the elongated member 40. Although not illustrated, the inner insertion tube 90 and the outer fitting tube 70 are arranged to coaxially extend up to the hand operation unit 50.

The inner insertion tube 90 and the outer fitting tube 70 are composed of flexible members made of a soft material or an elastic material. Examples of the constituent material thereof include polyvinyl chloride, polyvinyl acetate, a polyethylene-vinyl acetate copolymer, polyethylene, polypropylene, cyclic polyolefin, polybutadiene, polyurethane, a polyurethane elastomer, polyimide, polyamide, a polyether block amide copolymer, polyester, a polyester elastomer, a silicone resin, fluorine resins such as polytetrafluoroethylene, and a polyetheretherketone resin.

As illustrated in FIGS. 3A and 3B, each of the joint members 60 can include a shoulder 63 which is inclined at a predetermined angle, a head 62 which is located on the tip side of the shoulder 63, a flat part 64 which is continuous from the shoulder 63 toward the base end side, and a bottom 65 which is located on the base end side of the flat part 64.

Each of the joint members 60 has a through hole 61 which is formed from the bottom 65 through the head 62 along the axial direction of the elongated member 40. The through holes 61 of the joint members 60 form a lumen 69 on the elongated member 40, the lumen 69 penetrating the joint members 60 arranged side by side (refer to FIG. 6). Further, a lumen 91 is formed on the inner insertion tube 90. The lumen 91 is arranged coaxially with the through holes 61 of the joint members 60. Accordingly, the elongated member 40 has a lumen 95 which is formed from the through hole 13a of the base 13 to which the first open-close member 11 and the second open-close member 12 are attached, the lumen 69 formed from the through holes 61 of the respective joint members 60, and the lumen 91 of the inner insertion tube 90, the through hole 13a, the lumen 69, and the lumen 91 being continuous with each other. The lumen 95 can be used for inserting a guide wire through the lumen for guiding the medical instrument 100 to a predetermined region of a living body or an imaging device, for example, an endoscope, or used for delivering a cleaning solution, for example, saline, or sucking a living tissue liquid.

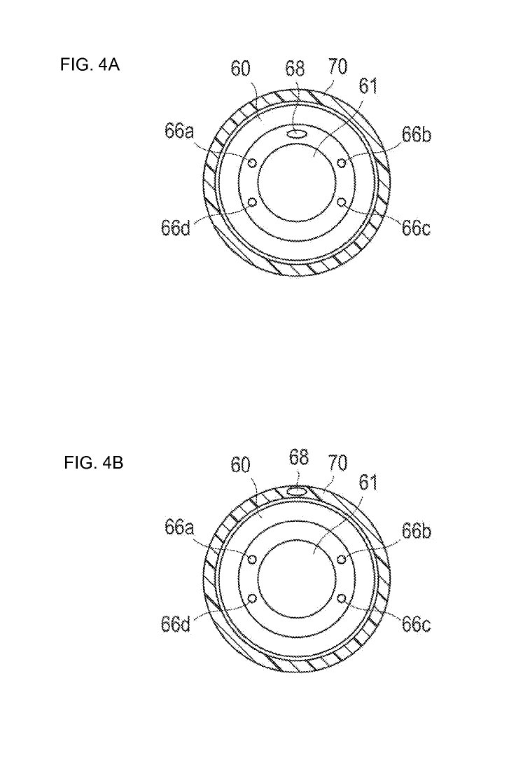

A predetermined hole 15 may be formed on the first open-close member 11 and the second open-close member 12 (refer to FIG. 2A). The hole 15 can enable a guide wire or an imaging device to be sent to the tip side of the first open-close member 11 and the second open-close member 12 when the first open-close member 11 and the second open-close member 12 are in a closed state. The hand operation unit 50 may include a port 57 for introducing a guide wire or various imaging means (refer to FIG. 1). For example, as illustrated in FIG. 4A, holes 68 which form a lumen for inserting, for example, a guide wire through the lumen may be formed on wall thickness parts of the respective joint members 60 separately from the lumen 95 of the elongated member 40. Alternatively, as illustrated in FIG. 4B, a hole 68 which forms a lumen for inserting, for example, a guide wire through the lumen may be formed on a wall thickness part of the outer fitting tube 70.

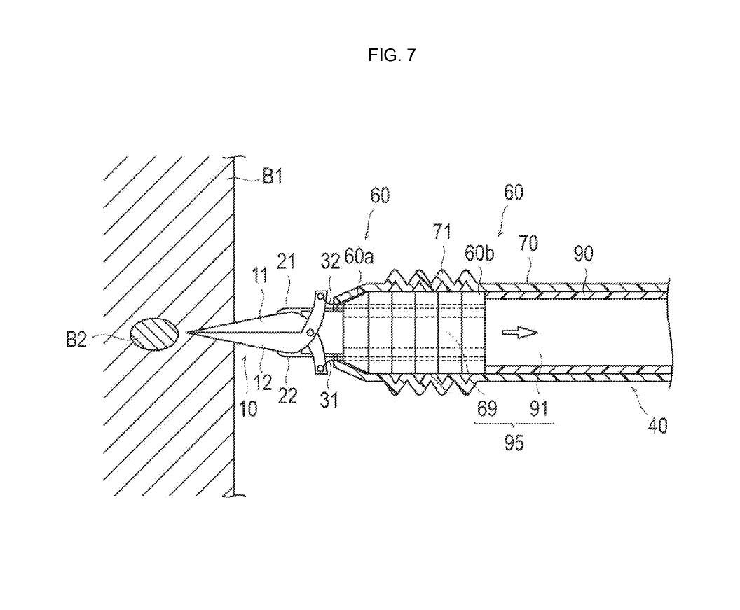

Referring to FIGS. 3A and 3B, the through hole 61 of each of the joint members 60 can be formed in a predetermined groove shape so as to introduce into the through hole 61, a predetermined region of an adjacent joint member 60 including the head 62 and the shoulder 63. As illustrated in FIG. 6, the joint members 60 can be arranged side by side in such a state that a predetermined region on the tip side of each of the joint members 60 is introduced into the through hole 61 of another joint member 60 adjacent thereto. The elongated member 40 can take a first form which is bendable in a direction intersecting with the axial direction when the joint members 60 are arranged side by side without having contact with each other, but with gaps therebetween. As illustrated in FIG. 7, the elongated member 40 can take a second form having rigidity when the distance between the joint members 60 is reduced in the axial direction so that the joint members 60 are arranged side by side having contact with each other. As will be described below, an operation of switching the elongated member 40 between the first form and the second form (switching operation) can be performed by an operation of pulling the first push-pull member 21 and the second push-pull member 22 or by an operation of pushing back the first push-pull member 21 and the second push-pull member 22 to move in a direction opposite to the pulling direction. A bending angle of the elongated member 40 in the first form is determined depending on an inclination angle of the shoulder 63 of each of the joint members 60. Thus, the bending angle of the elongated member 40 can be adjusted by changing the inclination angle of the shoulder 63.

In accordance with an exemplary embodiment, five joint members 60 can be used in the medical instrument 100. However, the number of joint members 60 is not particularly limited, and may be changed. Further, the material of the joint members 60 is not particularly limited, and may be, for example, a predetermined metal or resin material.

As illustrated in FIGS. 3A and 3B, each of the joint members 60 may have an insertion hole 66a through which the first push-pull member 21 is inserted, an insertion hole 66b through which the second push-pull member 22 is inserted, an insertion hole 66c through which the first auxiliary push-pull member 31 is inserted, and an insertion hole 66d through which the second auxiliary push-pull member 32 is inserted.

The joint members 60 can be coupled to each other through the push-pull members which are inserted through the insertion holes 66a, 66b, 66c, and 66d. Among the joint members 60, a joint member 60a which is located nearest to the tip (refer to FIG. 6) can be fixed to the first push-pull member 21 and the second push-pull member 22, and the other joint members 60 are not fixed to the first push-pull member 21 and the second push-pull member 22 in order to enable the switching operation of the elongated member 40 to be driven by the pulling operation of the push-pull members 21 and 22 as will be described below.

In accordance with an exemplary embodiment, the push-pull members inserted through the respective insertion holes 66a, 66b, 66c, and 66d of the joint member 60b which is located nearest to the base end illustrated in FIG. 6 may be arranged to directly extend up to the hand operation unit 50 through the lumen 95 of the elongated member 40, or may be arranged to extend up to the hand operation unit 50 through the inside of a wall thickness part of the inner insertion tube 90, a space between the inner insertion tube 90 and the outer fitting tube 70, or the inside of the wall thickness part of the outer fitting tube 70.

The first push-pull member 21 inserted through the insertion holes 66a is movable along the axial direction of the elongated member 40 while being coupled to each of the joint members 60 and the first open-close member 11. The second push-pull member 22 inserted through the insertion holes 66b is movable along the axial direction of the elongated member 40 while being coupled to each of the joint members 60 at a position that differs from the position to which the first open-close member 11 is coupled. In addition, the second push-pull member 22 being coupled to the second open-close member 12. The first auxiliary push-pull member 31 inserted through the insertion holes 66c and the second auxiliary push-pull member 32 inserted through the insertion holes 66d are also movable along the axial direction of the elongated member 40.

Next, the relationship between the pulling operation of the push-pull members, the opening-closing operation of the gripper, and the switching operation of the elongated member in the medical instrument 100 will be described.

FIG. 6 illustrates the medical instrument 100 with the gripper 10 in a closed state and the elongated member 40 in the first form bendable in the direction intersecting with the axial direction. As illustrated, when the distance between the joint members 60 is maintained at a predetermined distance that is sufficient to help prevent movement of each of the joint members 60 from being restricted, the entire elongated member 40 takes the bendable first form. As illustrated in FIG. 7, when the first push-pull member 21 and the second push-pull member 22 are pulled toward the base end side from the state illustrated in FIG. 6, the joint member 60a which is located nearest to the tip and fixed to the first push-pull member 21 and the second push-pull member 22 moves toward the base end side, and the other joint members 60 also move toward the base end side along with the movement of the joint member 60a. When the joint members 60 move toward the base end side by a predetermined distance, the joint member 60b which is located nearest to the base end abuts against the inner insertion tube 90, which helps restrict the movement of each of the joint members 60. When the distance between the joint members 60 is reduced to a distance that allows the joint members 60 to come into contact with each other, the elongated member 40 can be compressed along the axial direction and brought into the rigid second form in which the compressed state is maintained.

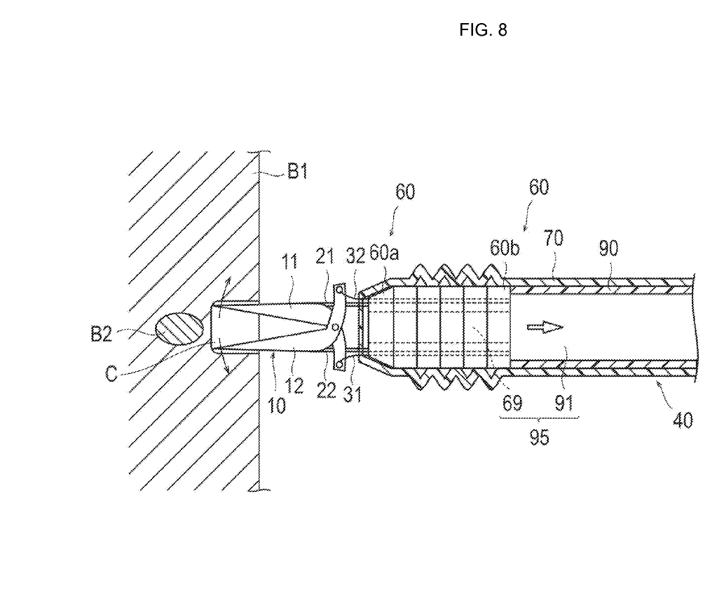

As illustrated in FIGS. 7 and 8, when the first push-pull member 21 and the second push-pull member 22 are further pulled after the elongated member 40 has been brought into the second form, the first open-close member 11 and the second open-close member 12 move away from each other in response to the pulling operation to thereby open the gripper 10 (first operation). In order to first perform the operation of switching the elongated member 40 from the first form to the second form and then perform the operation of opening the gripper 10, for example, a frictional force needed for opening and closing the gripper 10 is appropriately adjusted so that a pulling amount needed for opening the gripper 10 becomes larger than a pulling amount needed for starting the switching operation.

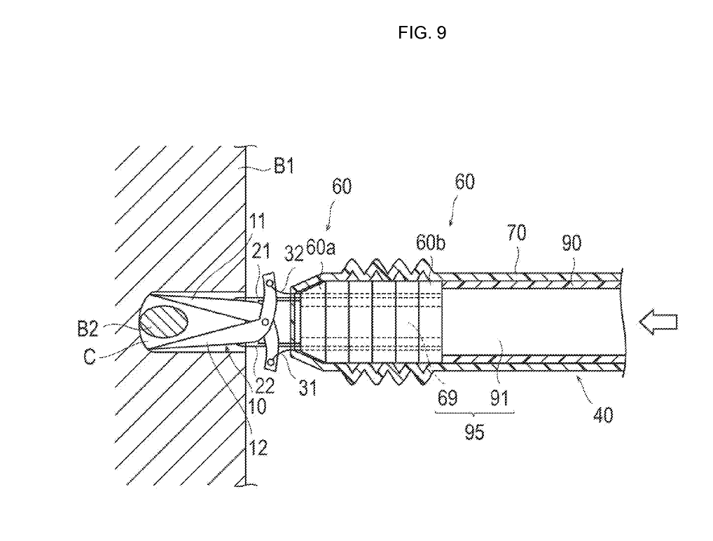

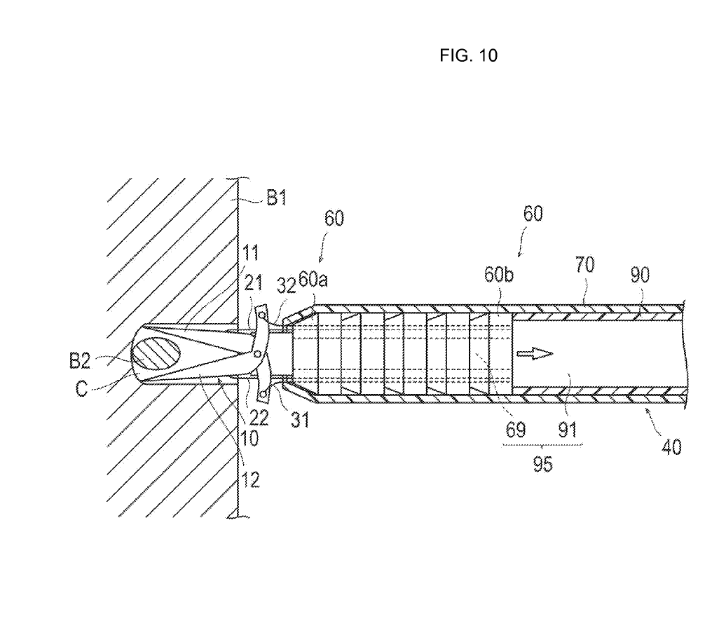

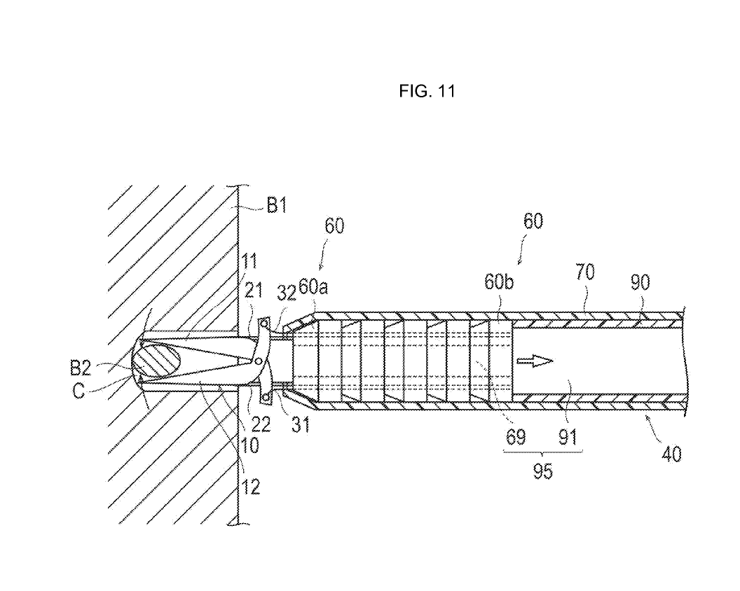

As illustrated in FIGS. 9 and 10, when the first push-pull member 21 and the second push-pull member 22 are pushed back to move in the direction opposite to the pulling direction with the gripper 10 open, the distance between the joint members 60 is increased to thereby switch the elongated member 40 from the rigid second form compressed in the axial direction to the bendable first form. Then, as illustrated in FIG. 11, when the first push-pull member 21 and the second push-pull member 22 are allowed to further move in the direction opposite to the pulling direction, the first open-close member 11 and the second open-close member 12 move closer to each other to thereby close the gripper 10 (second operation).

As illustrated in FIG. 7, a displacement preventer 71 may be provided in the medical instrument 100. When the elongated member 40 is switched from the first form to the second form, the displacement preventer 71 is deformed to project in the direction intersecting with the axial direction of the elongated member 40 to help prevent the elongated member 40 from being displaced from a predetermined position. The displacement preventer 71 may be composed of a part of the outer fitting tube 70. In the outer fitting tube 70 which is arranged to cover the joint members 60, regions that face the flat parts 64 of the respective joint members 60 are joined to the joint members 60, and the other regions are not joined to the joint members 60 (refer to FIG. 3A). Thus, when the first push-pull member 21 and the second push-pull member 22 are pulled and the joint members 60 thereby move toward the base end side, the regions that are not joined to the outer fitting tube 70 are compressed along the axial direction and deformed into a bellows shape as illustrated. The regions deformed in a bellows shape partially increase an outer diameter of the elongated member 40, which can act as a holding force for holding the elongated member 40 to the surroundings.

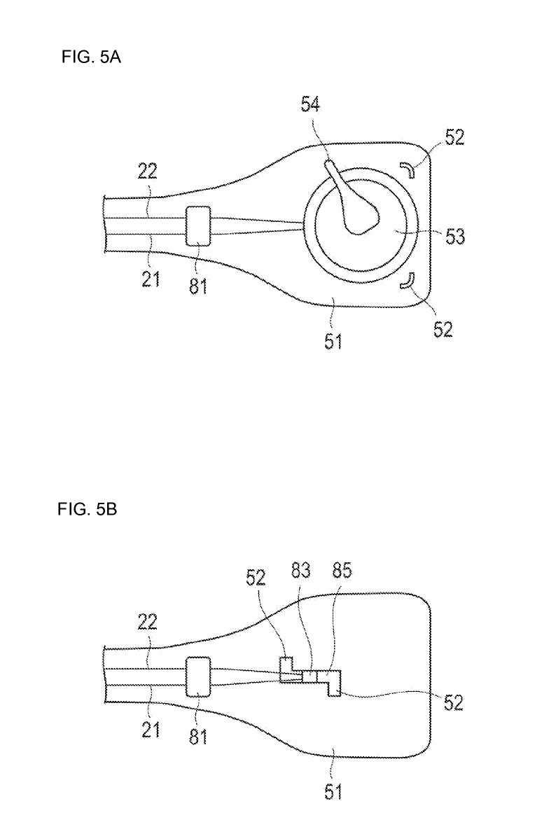

The hand operation unit 50 provided in the medical instrument 100 is capable of operating the movement of the first push-pull member 21 and the second push-pull member 22. The operation for each of the push-pull members 21 and 22 may be performed through a pulling mechanism 51 which can be arranged on the hand operation unit 50 as illustrated in FIG. 5A or FIG. 5B.

A pulling mechanism 51 illustrated in FIG. 5A is provided with a rotary member 53 to which the push-pull members 21 and 22 joined together by a predetermined fastener 81 is attached and a handle 54 for driving a rotation operation of the rotary member 53. Rotating the handle 54 in the clockwise direction can wind up or pull the push-pull members 21 and 22 so as to be pulled toward the base end side. In accordance with an exemplary embodiment, rotating the handle 54 in the counterclockwise direction can push the push-pull members 21 and 22 toward the tip side.

An open-close holder 52 may be provided in the hand operation unit 50. The open-close holder 52 can restrict the movement of the first push-pull member 21 and the second push-pull member 22 by holding the gripper 10 in an open state or a closed state. The open-close holder 52 may be composed of, for example, a stopper which fixes the handle 54 at a position for opening the gripper 10 or a position for closing the gripper 10 to thereby restrict the rotation operation. Further, a stopper function with which a part of the handle 54 can be fitted may be provided in the open-close holder 52 so that an operator who uses the medical instrument 100 can recognize that the gripper 10 has been opened or closed from the feeling of his/her hand.

A pulling mechanism 51 illustrated in FIG. 5B can include a slide member 83 to which the push-pull members 21 and 22 joined together by a predetermined fastener 81 is attached and a groove 85 within which the slide member 83 can slidably move. Moving the slide member 83 toward the base end side along the groove 85 can pull the push-pull members 21 and 22 toward the base end side. In accordance with an exemplary embodiment, moving the slide member 83 toward the tip side along the groove 85 can push back the push-pull members 21 and 22 toward the tip side. The open-close holder 52 can be composed of hooking parts formed on the base end side and the tip side of the groove 85. Hooking the slide member 83 on either one of the hooking parts can restrict the movement of the slide member 83, which can hold the gripper 10 in an open state or a closed state.

The pulling mechanism 51 having the form illustrated in FIG. 5A is employed in the medical instrument 100. In accordance with an exemplary embodiment, the pulling mechanism 51 having the form illustrated in FIG. 5B can be employed in the medical instrument 100. The arrangement position of the pulling mechanism 51 is not limited to the position illustrated in FIG. 1, and may be appropriately changed to a place in which the pulling mechanism 51 can be easily used. Further, in addition to the above configuration, each of the auxiliary push-pull members 31 and 32 may be connected to the pulling mechanism 51, and, for example, a pulling operation may be performed by the same operation as that performed with respect to the push-pull members 21 and 22.

Next, an application example of the medical instrument according to the embodiment will be described.

As set forth below, an example of a procedure of collecting a lesion (living tissue) B2, for example, a node formed on a living organ B1 in a respiratory region, for example, the bronchus will be described. However, a living organ as an application object and each step of the procedure are not limited thereto.

As illustrated in FIG. 6, the medical instrument 100 with the gripper 10 in a closed state and the elongated member 40 in the first form is introduced into a predetermined region of a living body. At this point, the elongated member 40 in the bendable first form can enable a relatively smooth introducing operation. The introducing operation may be performed, for example, using imaging means such as an endoscope and a bronchoscope or a guide wire.

As illustrated in FIG. 7, the push-pull members 21 and 22 are pulled to switch the elongated member 40 from the first form to the second form. At this point, the elongated member 40 may be held on, for example, a channel of the endoscope or a lumen of the living body by the displacement preventer 71.

Then, the elongated member 40 is pushed into the wall of the living organ B1 so that a tip part of the gripper 10 is inserted into the wall. The insertion is performed in the second form having high pushability in which the tip part of the elongated member 40 is compressed in the axial direction. Therefore, the gripper 10 can be inserted relatively smoothly into the wall. Further, the tapered shape of the tip of the gripper 10 can help enable an efficient insertion operation.

At this point, when the wall is relatively soft, the gripper 10 may be inserted in an open state. In the insertion, the gripper 10 may be pushed into the wall while rotating the gripper 10 by an operation by hand.

As illustrated in FIG. 8, the push-pull members 21 and 22 are pulled to open the gripper 10. Opening the gripper 10 forms a cavity C within the wall of the living organ. When tissue within the wall is hard and the gripper 10 cannot be opened merely by pulling the push-pull members 21 and 22, the gripper 10 may be opened using the auxiliary push-pull members 31 and 32.

As illustrated in FIG. 9, the gripper 10 is further pushed into the wall of the living organ B1. Also at this point, since the elongated member 40 is in the second form in which the tip part thereof is compressed in the axial direction, the gripper 10 can be smoothly pushed within the living organ B1.

After the gripper 10 has been arranged to sandwich the lesion B2 as a collection object as illustrated in FIG. 10, the push-pull members 21 and 22 are pushed back in the direction opposite to the pulling direction. This operation switches the elongated member 40 from the second form to the first form.

As illustrated in FIG. 11, the push-pull members 21 and 22 are further pushed back in the direction opposite to the pulling direction to close the gripper 10. Accordingly, the lesion B2 can be gripped by the gripper 10. Even when living tissue is hard or when living tissue is adhered with a relatively strong adhesive force, the living tissue can be cut when the blades 19 are formed on the gripping surfaces 18a and 18b of the gripper 18. Therefore, the living tissue can be reliably collected. Then, the medical instrument 100 is taken out of the living body while gripping the lesion B2.

As described above, the medical instrument according to the present exemplary embodiment is capable of providing a method for collecting living tissue using a medical instrument that is provided with an elongated member having a plurality of joint members arranged side by side in the axial direction, a gripper that is freely opened and closed, a first push-pull member and a second push-pull member each coupled to the joint members and the gripper, the first and second push-pull members being movable along the axial direction. In accordance with an exemplary embodiment, the collection method can include (i) introducing the medical instrument into a living body with the elongated member taking a first form bendable in a direction intersecting with the axial direction in which the joint members are arranged side by side with gaps therebetween in the axial direction, (ii) pulling the first push-pull member and the second push-pull member to reduce the distance between the joint members in the axial direction to bring the elongated member into a second form having rigidity, (iii) pulling the first push-pull member and the second push-pull member with the elongated member taking the second form to drive a first operation for opening or closing the gripper, (iv) pushing back the first push-pull member and the second push-pull member to move in a direction opposite to the pulling direction after the first operation is driven to increase the distance between the joint members to bring the elongated member into the first form, and (v) then pushing back the first push-pull member and the second push-pull member to move in the direction opposite to the pulling direction to drive a second operation that is opposite to the first operation for opening or closing the gripper.

In accordance with an exemplary embodiment, the collection method can include inserting the gripper into the wall of the living organ after step (ii) and performing the first operation with the gripper inserted into the wall of the living organ in step (iii).

The first operation opens the gripper and the second operation closes the gripper. The collection method can include gripping a predetermined region of living tissue in step (v).

As described above, according to the medical instrument 100 of the present exemplary embodiment, the elongated member 40 can be switched between the bendable first form and the second form having rigidity in the axial direction and also to open and close the gripper 10 by performing the operation of pulling the first push-pull member 21 and the second push-pull member 22 and the operation of moving the first push-pull member 21 and the second push-pull member 22 in the direction opposite to the pulling direction. Thus, it is not necessary to provide an operation member for operating the opening-closing operation of the gripper 10 in addition to the push-pull members 21 and 22 for operating the switching of the elongated member 40 between the bendable state and the rigid state. Accordingly, the device configuration can be prevented from becoming complicated. Thus, the medical instrument 100 can have excellent operability, and the medical instrument 100 can be capable of collecting living tissue present within the wall of a living organ without deteriorating the deliverability of the gripper 10 inside a living body.

When the first auxiliary push-pull member 31 and the second auxiliary push-pull member 32 each for driving the opening-closing operation of the gripper 10 are provided, the gripper 10 can be opened and closed by operating the first auxiliary push-pull member 31 and the second auxiliary push-pull member 32. Therefore, the gripper 10 can be reliably opened and closed.

When each of the first open-close member 11 and the second open-close member 12 has a tapered shape tapered toward the tip side, the gripper 10 can be smoothly inserted into the wall of a living organ. Further, when the blades 19 are formed on the gripping surfaces 18a and 18b on which the open-close members 11 and 12 face each other, an object to be gripped within the wall by cutting the object with the blades 19 can be collected.

When the through holes 61 which penetrate the elongated member 40 in the axial direction are formed on the respective joint members 60, the lumen 69 which penetrates the joint members 60 arranged side by side on the elongated member 40 can be formed.

When the displacement preventer 71 is provided in the medical instrument 100, the elongated member 40 can be prevented from being displaced when the elongated member 40 is switched from the first form to the second form.

When the hand operation unit 50 has the open-close holder 52 which helps restrict the movement of the first push-pull member 21 and the second push-pull member 22 can hold the gripper 10 in an open state or a closed state, various operations while holding the gripper 10 in an open state or a closed state by the open-close holder 52 provided in the hand operation unit 50 can be performed.

When the first opening-closing operation opens the gripper 10 and the second opening-closing operation closes the gripper 10, the operation of pulling the push-pull members 21 and 22 is converted into the operation of opening the gripper 10. Thus, the gripper 10 can be opened with a stronger force than when the gripper 10 is opened by pushing back the push-pull members 21 and 22. Therefore, the gripper 10 can be reliably opened even when the gripper 10 is inserted into the wall of a living organ.

Further, the living tissue collecting medical instrument 100 can be suitable for collecting living tissue in a respiratory region.

The medical instrument according to the present disclosure is not limited to the configuration described in the above embodiment, and may be variously modified on the basis of the description of the claims.

For example, it is only required that at least one push-pull member be provided in each of the open-close members. Therefore, the number of push-pull members to be placed may be increased. Similarly, when the auxiliary push-pull members are used, the number of auxiliary push-pull members to be placed may be increased.

There has been described the embodiment in which the first operation opens the gripper 10 and the second operation closes the gripper 10 as operations of the gripper 10. However, each of the operations may be an operation opposite to the above operation. For example, the gripper 10 may be closed by pulling the push-pull members, and opened by pushing back the push-pull members to move in the direction opposite to the pulling direction.

The detailed description above describes a medical instrument. The disclosure is not limited, however, to the precise embodiments and variations described. Various changes, modifications and equivalents can effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. A medical instrument comprising:
an elongated member having a plurality of joint members arranged side by side in an axial direction, the elongated member taking a first form that is bendable in a direction intersecting with the axial direction when the joint members are arranged with gaps therebetween in the axial direction and taking a second form having rigidity when a distance between the joint members is reduced in the axial direction to be narrower than the distance in the first form;
a gripper arranged on one end side of the elongated member, the gripper having a first open-close member and a second open-close member, the first open-close member and the second open-close member being movable closer to each other or away from each other;
a first push-pull member coupled to the joint members and the first open-close member, the first push-pull member being movable along the axial direction of the elongated member; and
a second push-pull member coupled to the joint members at positions differing from positions at which the first open-close member is coupled and to the second open-close member, the second push-pull member being movable along the axial direction of the elongated member, wherein
the elongated member takes the second form by pulling the first push-pull member and the second push-pull member in the axial direction with the elongated member taking the first form to reduce the distance between the joint members,
a first operation for opening or closing the gripper is driven by pulling the first push-pull member and the second push-pull member with the elongated member taking the second form,
the elongated member takes the first form by pushing back the first push-pull member and the second push-pull member to move in a direction opposite to the pulling direction after the first operation of the gripper has been driven to increase the distance between the joint members, and
a second operation that is opposite to the first operation for opening or closing the gripper is driven by pushing back the first push-pull member and the second push-pull member to move in the direction opposite to the pulling direction with the elongated member taking the first form.

2. The medical instrument according to claim 1, further comprising:
a first auxiliary push-pull member coupled to the first open-close member, the first auxiliary push-pull member being movable along the axial direction of the elongated member; and
a second auxiliary push-pull member coupled to the second open-close member, the second auxiliary push-pull member being movable along the axial direction of the elongated member, wherein
an opening-closing operation of the gripper can be driven by pulling the first auxiliary push-pull member and the second auxiliary push-pull member along the axial direction or pushing back the first auxiliary push-pull member and the second auxiliary push-pull member to move in a direction opposite to the pulling direction.

3. The medical instrument according to claim 1, wherein each of the first open-close member and the second open-close member has a tapered shape tapered toward a tip side, and blades are formed on gripping surfaces on which the first and second open-close members face each other.

4. The medical instrument according to claim 1, wherein the joint members have through holes penetrating the elongated member in the axial direction.

5. The medical instrument according to claim 1, further comprising:
a displacement preventer that is deformed to project in a direction intersecting with the axial direction of the elongated member when the elongated member is switched from the first form to the second form to prevent the elongated member from being displaced from a predetermined position.

6. The medical instrument according to claim 1, further comprising:
a hand operation unit arranged on the other end side of the elongated member, the hand operation unit being capable of operating movement of the first push-pull member and the second push-pull member, wherein
the hand operation unit has an open-close holder restricting the movement of the first push-pull member and the second push-pull member to hold the gripper in an open state or a closed state.

7. The medical instrument according to claim 1, wherein
the first operation moves the first open-close member and the second open-close member away from each other to open the gripper, and
the second operation moves the first open-close member and the second open-close member closer to each other to close the gripper.

8. The medical instrument according to claim 1, wherein the medical instrument is a living tissue collecting medical instrument used for collecting living tissue in a respiratory region.

9. The medical instrument according to claim 2, wherein each of the first open-close member and the second open-close member has a tapered shape toward a tip side, and blades are formed on gripping surfaces on which the first and the second open-close members face each other.

10. The medical instrument according to claim 2, comprising:
a displacement preventer that is deformed to project in a direction intersecting with the axial direction of the elongated member when the elongated member is switched from the first form to the second form to prevent the elongated member from being displaced from a predetermined position.

11. The medical instrument according to claim 3, comprising:
a displacement preventer that is deformed to project in a direction intersecting with the axial direction of the elongated member when the elongated member is switched from the first form to the second form to prevent the elongated member from being displaced from a predetermined position.

12. The medical instrument according to claim 2, comprising:
a hand operation unit arranged on the other end side of the elongated member, the hand operation unit being capable of operating movement of the first push-pull member and the second push-pull member, wherein
the hand operation unit has an open-close holder restricting the movement of the first push-pull member and the second push-pull member to hold the gripper in an open state or a closed state.

13. The medical instrument according to claim 3, comprising:
a hand operation unit arranged on the other end side of the elongated member, the hand operation unit being capable of operating movement of the first push-pull member and the second push-pull member, wherein
the hand operation unit has an open-close holder restricting the movement of the first push-pull member and the second push-pull member to hold the gripper in an open state or a closed state.

14. The medical instrument according to claim 5, comprising:
a hand operation unit arranged on the other end side of the elongated member, the hand operation unit being capable of operating movement of the first push-pull member and the second push-pull member, wherein
the hand operation unit has an open-close holder restricting the movement of the first push-pull member and the second push-pull member to hold the gripper in an open state or a closed state.

15. The medical instrument according to claim 2, wherein
the first operation moves the first open-close member and the second open-close member away from each other to open the gripper, and
the second operation moves the first open-close member and the second open-close member closer to each other to close the gripper.

16. The medical instrument according to claim 3, wherein
the first operation moves the first open-close member and the second open-close member away from each other to open the gripper, and
the second operation moves the first open-close member and the second open-close member closer to each other to close the gripper.

17. The medical instrument according to claim 5, wherein
the first operation moves the first open-close member and the second open-close member away from each other to open the gripper, and
the second operation moves the first open-close member and the second open-close member closer to each other to close the gripper.

18. The medical instrument according to claim 6, wherein
the first operation moves the first open-close member and the second open-close member away from each other to open the gripper, and
the second operation moves the first open-close member and the second open-close member closer to each other to close the gripper.
